(19) United States
(12) Patent Application Publication
(10) Pub. No.: US 2024/0387026 A1
(43) Pub. Date: Nov. 21, 2024

(54) Systems and Methods for Managing Caregiver Overload

(71) Applicant: Stryker Corporation, Kalamazoo, MI (US)

(72) Inventors: Rajiv Puranik, Cupertino, CA (US); Benjamin Kanter, Escondido, CA (US); Akkireddy Gunta, Bangalore (IN); Heidi Gianella, San Jose, CA (US)

(21) Appl. No.: 18/682,362

(22) PCT Filed: Aug. 10, 2022
(86) PCT No.: PCT/US2022/039920

§ 371 Date: Feb. 8, 2024

Related U.S. Application Data
(60) Provisional application No. 63/231,460, filed on Aug. 10, 2021.

(51) Int. Cl. G16H 40/20

(52) U.S. Cl. CPC G16H 40/20

(57) ABSTRACT

Embodiments include methods, devices, systems, and non-transitory process-readable storage media for managing an overload condition of a care provider. Some embodiments may include monitoring communications sent to a care provider communication device to identify a plurality of overload factors, determining whether the plurality of overload factors meets an overload condition for the care provider, and performing a mitigation operation in response to determining that the plurality of overload factors meets the overload condition for the care provider.

### RELATED APPLICATIONS

This application claims the benefit of priority to U.S. Provisional Application No. 63/231,460 entitled “Systems And Methods For Managing Caregiver Overload” filed Aug. 10, 2021, the entire contents of which are hereby incorporated by reference for all purposes.

### BACKGROUND

Wireless communication systems are increasingly utilized to facilitate coordination among workgroups in a variety of environments. Wireless communication systems have proven highly effective in hospitals and other healthcare environments, because mobile communicators enable rapid communication among doctors, nurses, and other care team staff who provide time-critical patient care and need to respond to medical sensor alarms, calls and pages from other care team staff, and patient requests, in addition to the routine daily administrative messages and interruptions.

However, as an unintended consequence of the instant access such improved communication capabilities provide, modern healthcare environments can be overwhelming and stressful for care providers who wear such mobile communicators. A constant torrent of messages, calls, alerts, pages, voice calls, mass messages, and public announcements demanding user attention from the mundane to time-critical emergencies involving the life or health of patients can lead to emotional stress and overload, which may contribute to medical errors and care provider burnout.

### SUMMARY

Various embodiments provide methods, systems, wireless communication devices, and non-transitory process-readable storage media for enabling a communication system to identify when a user has become or is becoming overloaded and to perform a mitigation operation to alleviate the user overload condition.

Various embodiments include methods and network computing devices configured to perform methods that include monitoring communications sent to a care provider communication device to identify a plurality of overload factors, determining whether the plurality of overload factors meets an overload condition for the care provider, and performing a mitigation operation in response to determining that the plurality of overload factors meets the overload condition for the care provider.

In some embodiments, the plurality of overload factors may include one or more interrupt events, wherein each interrupt event is associated with a severity metric. Some embodiments may include associating with each interrupt event a congruity metric indicating a level of congruity of each interrupt event relative to one or more other interrupt events. Some embodiments may include associating with each interrupt event an acuity metric indicating a level of acuity of the interrupt event. In some embodiments, monitoring communications sent to a care provider communication device to identify a plurality of overload factors may include determining response times for receiving a response from a care provider communication device to communications. In some embodiments, monitoring communications to a care provider communication device to identify care provider a plurality of overload factors may include monitoring communications that are ignored or declined by the care provider communication device.

In some embodiments, monitoring communications to a care provider communication device to identify a plurality of overload factors may include monitoring communications that are responded to with a do not disturb instruction. In some embodiments, monitoring communications to a care provider communication device to identify a plurality of overload factors may include monitoring a number of communications that have not been answered. In some embodiments, monitoring communications to a care provider communication device to identify a plurality of overload factors may include monitoring how long the care provider has been working. In some embodiments, monitoring communications sent to a care provider communication device to identify a plurality of overload factors may include determining intervals between communications.

In some embodiments, determining whether the plurality of overload factors meets an overload condition for the care provider may include determining whether the plurality of overload factors modulated by one or more care provider skill factors meets the overload condition. In some embodiments, determining whether the plurality of overload factors meets an overload condition for the care provider may include determining whether the plurality of overload factors modulated by a health condition of the care provider meets the overload condition. In some embodiments, determining whether the plurality of overload factors meets an overload condition for the care provider may include applying the plurality of overload factors to an overload condition model that is configured to provide as an output whether the plurality of overload factors meets the overload condition.

In some embodiments, applying the plurality of overload factors to an overload condition model that is configured to provide as an output whether the plurality of overload factors meets the overload condition may include applying one or more care provider skill factors to the overload condition model, wherein the overload condition model is configured to provide as an output whether the plurality of overload factors modulated by the skill factors meets the overload condition. In some embodiments, applying the plurality of overload factors to an overload condition model that is configured to provide as an output whether the plurality of overload factors meets the overload condition may include applying a health condition of the care provider to the overload condition model, wherein the overload condition model is configured to provide as an output whether the plurality of overload factors modulated by the health condition of the care provider meets the overload condition.

In some embodiments, performing a mitigation operation may include sending information to a network computing device to reduce a frequency of communications to the care provider. In some embodiments, performing a mitigation operation may include sending information to a network computing device to adjust one or more tasks assigned to the care provider.

Further embodiments include a network computing device having a processor configured with processor-executable instructions to perform operations of any of the methods summarized above. Further embodiments include a non-transitory processor-readable medium on which is stored processor-executable instructions configured to cause a processor of a network computing device to perform operations of any of the methods summarized above. Further embodiments include a network computing device having means for performing functions of any of the methods summarized above.

### BRIEF DESCRIPTION OF THE DRAWINGS

The accompanying drawings, which are incorporated herein and constitute part of this specification, illustrate exemplary embodiments of the invention, and together with the general description given above and the detailed description given below, serve to explain the features of the invention.

FIG. 1 is a component block diagram of a communication system suitable for use in various embodiments.

FIG. 2 is a conceptual block diagram illustrating a workflow suitable for use in various embodiments.

FIG. 3 is a process flow diagram illustrating a method that may be performed by a processor of a computing device for managing a user overload condition 300 according to various embodiments.

FIGS. 4A-4C are process flow diagrams illustrating operations that may be performed by a processor of a network computing device as part of the method for managing a user overload condition according to various embodiments.

FIGS. 5A and 5B are process flow diagrams illustrating operations that may be performed by a processor of a network computing device as part of the method for managing a user overload condition according to various embodiments.

FIG. 6 illustrates a communication device suitable for use in various embodiments.

FIG. 7 illustrates a server device suitable for use in various embodiments.

### DETAILED DESCRIPTION

Various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

Various embodiments provide methods, systems, wireless communication devices, and non-transitory process-readable storage media for enabling a communication enable systems that provide ubiquitous instantaneous communications to users, particularly users in stressful work conditions, to recognize when communications delivered to communication device user, such as a medical care provider, are overwhelming or “flooding” the user. By automatically recognizing when a communication device user is, is becoming, or is predicted to become overloaded or “flooded” by messages, alarms, pages, and calls within a period of time, the system can take an action to aid the user and/or avoid further overloading the user, which could lead to mistakes and emotional distress.

As used herein, the term “communication” refers to any of a variety of messages that the communication device may receive, including phone calls (e.g., voice calls), email messages, text messages, alert messages, notifications, tasks, action assignment messages, and the like.

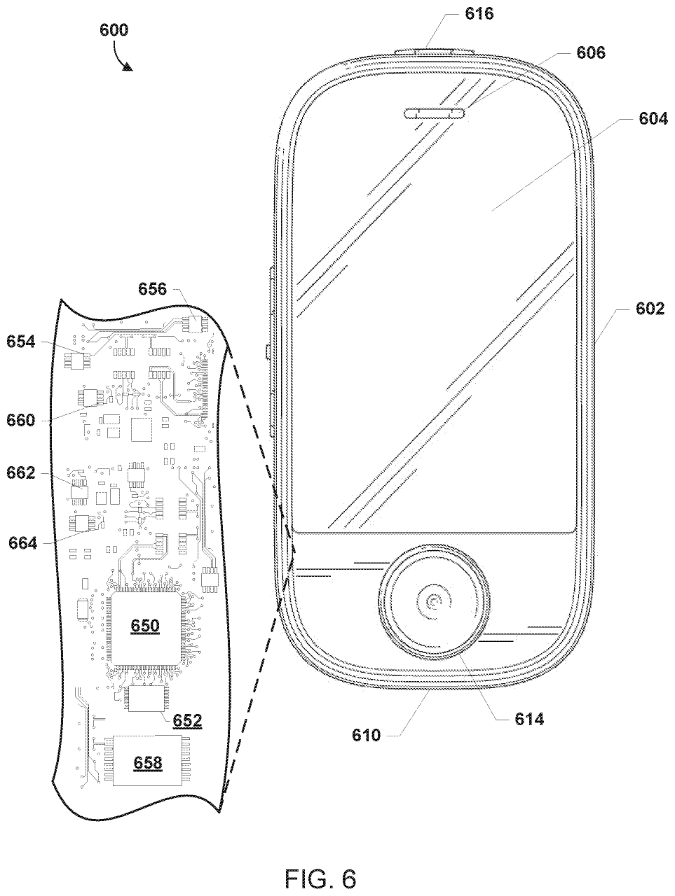

The term “communication device” is used herein to refer to an electronic device equipped with at least a processor and an interface configured to enable communication with a communication system or network. Some embodiments may include a wireless communication device equipped with at least a processor and a transceiver configured to support wireless communications with a wireless local area network (WLAN). Examples of communication devices include mobile devices, such as communicators for use within a hospital or other healthcare environments, and stationary or semi-stationary communication devices, such as terminals, surface-mounted communication devices, and other similar communication devices. In various embodiments, communication devices may be configured with memory or storage as well as networking capabilities, such as network transceiver(s) and antenna(s) configured to establish a WLAN connection with an access point. Communication devices may also include voice communications badge devices, an example of which is illustrated in FIG. 6.

In the modern world, professionals are increasingly always-connected, with smartphones and other wireless communication devices providing instant and continuous access to a variety of communications. For individuals engaged in stressful work, particularly those involving health and safety of themselves and others, a constant barrage of interruptions, alarms, calls, messages, etc. can lead to conditions in which the user may become flooded with too many interruptions, too much information, too many demands with insufficient time to process the information and demands on the user's time. There are limits to how many different sources of information individuals can process at one time, and how many interruptions individuals may respond to within a period of time before losing focus or feeling overwhelmed. When the consequences of mistakes or inattention could be life threatening or catastrophic, as with an airline pilot, doctor or nurse, power plant operator, or other high-skill/high-risk professional, the potential importance of each alarm, communication or interruption may make such limits more challenging. When individuals engaged in stressful work become overwhelmed or “flooded,” errors and accidents are more likely, and over the long term burnout may result.

Monitoring the emotional status of professionals, such as care providers, is challenging. Many professionals expect of themselves, or feel that they are expected to have, superhuman levels of stress tolerance. Consequently, asking professionals to self-report high levels of stress is notoriously unreliable for detecting whether anyone is overloaded. At best, a individuals may report being overloaded after a work shift is completed, too late to remedy the situation. At worst, co-workers or managers may not know that a care provider is stressed beyond tolerance until it is too late, such as after a mistake occurs, or the professional suffers some form of physical fatigue or emotional breakdown, or the care provider quits their job.

An example of the potential for overwhelming professionals with too many communications and interruptions exists in modern healthcare environments in which care providers are provided with personal communication devices connected to hospital communication systems. Care providers in such environments may receive a torrent of information and requests for their attention via their communication devices, and due to the critical nature of their duties such users cannot ignore a communication as the next interruption could be a life-or-death situation. Messages, calls, alerts, pages, voice calls, mass messages, and public announcements demanding care provider attention may range from the mundane human resources reminder to time-critical emergencies involving the life or health of patients. The more messages care providers receive per unit time (e.g., half hour, hour, shift, etc.), the more likely a care provider may become overloaded and emotionally stressed.

Various embodiments enable a computing device in a communication system (a “network computing device”) to identify when a communication device user has become or is becoming overloaded in real time (e.g., during a shift), and to perform a mitigation operation to alleviate the overload situation. Various embodiments have wide applicability in a variety of work environments in which users in stressful jobs are equipped with communication devices coupled to the communication system. For ease of description, various embodiments are described herein using the example of medical care providers, such as doctors, nurses and therapists in a hospital setting configured with a communication system in which the care providers are equipped with individual communication devices. However, the use of such examples is not intended to limit the scope of the claims unless specifically recited.

In various embodiments, the network computing device may monitor communications sent to a care provider communication device to identify a plurality of overload factors. As used herein, the term “overload factor” refers to any of a variety of events and conditions that may lead to user overload under various conditions, and include event-type factors such as communications received via a communication device, the frequency of communications and interruptions, the importance or criticality of communications, the familiarity or normality (or unfamiliarity or unusual) nature of the communications, etc., and individual factors such as individual roles or missions, time of day, time on shift, day of week, number and severity of interrupts already handled during the current shift, etc. The network computing device may determine whether the plurality of overload factors meets an overload condition for the care provider. In response to determining that the plurality of overload factors meet the overload condition for the care provider the network computing device may perform a mitigation operation. In some embodiments, the network computing device may include a device, service, or function implemented as part of a communication system in a manner that enables the network computing device to monitor communications to and from a care provider's communication device. In some embodiments, the network computing device may monitor communications to the care provider communication device over a period of time. In some embodiments, the network computing device may employ a sliding window for monitoring such communications.

As noted above, communications received by a communication device user, such as a care giver, may range from the mundane to time-critical emergencies (e.g., involving the life or health of patients), but the critical the communication, the more stress such an interruption may cause. Further, the potential stress induced by a given communication in an individual may depend upon the training and experience of the individual, as well as whether the nature of the communication is familiar to the individual. Thus, in some embodiments, the network computing device may be configured to dynamically determine the plurality of overload factors, and to dynamically determine whether the plurality of overload factors meets the overload condition in a manner that is individualized for each care provider. In this manner, the network computing device may be configured to perform an individualized detection of whether the care provider is overloaded, rather than applying a one-size-fits-all definition of an overload condition (e.g., defining an overload condition for an individual as ten or more events in any ten minute period).

In some embodiments, the mitigation operation may communicate the detection of the overload condition to a clinical workflow system so that the clinical workflow system can automatically make changes in the workflow affecting overloaded care providers. For example, a clinical workflow system may perform operations that move the overloaded care provider to a less stressful or quieter assignment for a period of time to enable that person to recover from the overload condition. For example, a clinical workflow system may limit or prevent additional task assignments communicated to the care provider communication device, limit or preventing unnecessary communications to the care provider communication device, and the like. In some embodiments, the clinical workflow system may perform a “load balancing” of tasks among a staff of care providers, monitoring each care providers' overload condition status in real time (e.g., during a shift).

In various embodiments, overload factors determined by the network computing device may include interrupt events. An “interrupt event” may include a message, a voice call, a notification, an alert, a page, a mass message, a public announcement, a task assignment or reassignment, or another communication sent to the care provider communication device. Further, in some embodiments, each interrupt event may be associated with a severity metric, which may be assigned by the network computing device based upon the nature, criticality or importance of each communication. In some embodiments, the severity metric may be based on content of the interrupt event (e.g., information in the interrupt event) or a context of the interrupt event. In some embodiments, the interrupt event context may include information related to the interrupt event, which may or may not be included in the interrupt event itself, and may be provided by another network computing device, or information associated with the interrupt event. As an example, an alert sent to an individual care provider that a patient is experiencing an emergency requiring immediate attention may be associated with a relatively high severity metric, because the recipient must respond and the consequences of the event could be grave. As another example, a broadcast message indicating a minor change in cafeteria service may be associated with a relatively low severity metric. In general, interrupt events associated with relatively high severity metrics may contribute more to the care provider's overload factors than interrupt events associated with relatively low severity metrics. In some embodiments, severity metrics may be used as weighting factors applied to event-based overload factors (e.g., alarms, messages, etc.) in determining weighted overload factors.

As noted above, messages and interrupts that are outside a user's normal activities or expertise (i.e., unfamiliar or unusual) may be more stressful, and thus contribute more to overload or “flooding” than familiar and routine messages and interrupts. In general, interrupt events that are incongruous or unusual tend to be more stressful for a care provider than routine tasks or occurrences. Thus, in some embodiments, the network computing device may associate each interrupt event with a congruity metric that indicates a level of congruity of each interrupt event relative to one or more other interrupt events. In some embodiments, the network computing device may determine the congruity metric based on the content and/or context of the interrupt event. In some embodiments, the congruity metric may be based on a care provider's role. For example, a message regarding a patient experiencing a cardiac emergency directed to a care provider assigned to work in a unit in which patients typically do not experience cardiac emergencies may be assigned a high level of incongruity (or a low level of congruity). As another example, an interrupt event for a relatively routine task or having routine information may be assigned a low level of incongruity (or a high level of congruity).

In some embodiments, the network computing device may associate each interrupt event with an acuity metric that indicates a level of acuity of the interrupt event. A level of acuity may an amount of care provider attention or effort required to address or attend to the interrupt event. For example, a high-acuity patient may require a nurse dedicated solely to that patient's care, whereas a low-acuity patient may share a nurse with other patients. So, a level of acuity may reflect a level of demand placed on the care provider by an interrupt event. Additionally or alternatively, the level of acuity of the interrupt event may reflect a complexity of care required to address or attend to the interrupt event. For example, a patient with complex care needs or a complex task will require a higher level of acuity than a simple task or a patient with simple or routine needs.

A communication system that manages communications to and from a user's communication device may monitor both communications to the user as well as responses to such communications provided by each user. In some situations, the time it takes each user to respond to a message that requires a response may provide insight into the demands being placed on the user at the time. Thus, in some embodiments, the network computing device may determine response times for receiving a response from the care provider communication device to communications sent to the communication device, and use this information to determine whether the plurality of overload factors meets an overload condition for the care provider based in part on the determined response times. For example, an overwhelmed care provider may take longer to respond to a new communication than a care provider who is not overwhelmed. Individual overload can be determined using baseline response latency of the particular care provider from responses to previous events. In some embodiments, the network computing device may compare timestamps of communications sent to the care provider's communication device and timestamps of responses received from the care provider's communication device. In some embodiments, the network computing device may determine that one or more response times for receiving a response from the care provider communication device exceeds a response time threshold. In some embodiments, the network computing device may determine a trend in response times from the care provider communication device. For example, the network computing device may determine that response times for responses from the care provider's communication device are getting longer and longer (e.g., the communication device is evincing less responsiveness over time). In some embodiments, the network computing device may determine that the trend in response times from the care provider communication device meets an overload condition, or contributes to an overload condition.

In some embodiments, the network computing device may keep track of when responses to certain communications are not received from the care provider's communication device. In some embodiments, the network computing device may identify such responses or non-responses in the plurality of overload factors. In some embodiments, the network computing device may monitor a number of communications that have not been answered. In some embodiments, as part of monitoring communications with the care provider's communication device, the network computing device may monitor communications that are ignored or declined by the care provider communication device. In some embodiments, the network computing device may monitor a change over time in the number of communications that have not been answered. For example, the network computing device may receive one or more responses from the care provider communication device declining to respond to an alert or notification about a patient condition. As another example, a number of unanswered communications may indicate a level of work being performed, a stress level, or a level of work engagement that a care provider is experiencing, and may meet an overload condition, or contribute to an overload condition. As another example, an increase over time in the number of unanswered communications may indicate that the care provider is over time becoming busier, or potentially more stressed, and may meet an overload condition, or may contribute to an overload condition.

In some embodiments, the network computing device may monitor communications that are responded to with a do not disturb instruction. For example, the network computing device determining that the care provider may be experiencing overload in response to receiving an instruction from the care provider communication device to limit or prevent communications to the care provider communication device.

In some embodiments, the network computing device may monitor how long the care provider has been working (e.g., on shift). As one example, a care provider at the beginning of their shift may be less readily overwhelmed than a care provider at the end of their shift. In some embodiments, the capacity of a care provider to respond to or handle tasks, communications, interrupt events, and the like may change non-linearly over time. In some embodiments, the network computing device may monitor how long the care provider has been working without a break. For example, a care provider may provide an indication via an input to a communication device that the care provider is taking a break, having a meal, and the like. In some embodiments, an amount of time that the care provider has been on shift, working without a break, and/or the like may meet an overload condition, or may contribute to an overload condition. In some embodiments, the network computing device may determine intervals between communications (e.g., sent to or from the care provider's communication device). If a care provider is receiving frequent communication or interrupt events, such condition may meet an overload condition, or may contribute to an overload condition.

In some embodiments, the network computing device may be configured to take into consideration a care provider's level of training, expertise, experience, and the like. In some embodiments, the network computing device may determine whether the plurality of overload factors modulated by one or more care provider skill factors meets the overload condition. In some embodiments, the network computing device may be configured to apply or incorporate one or more weight values representing one or more care provider skill factors to the plurality of overload factors. The skill factors may represent, for example, an ability of the care provider to handle a certain level of severity of a task or interrupt event, or a type of task or interrupt event. As one example, a trained cardiac care provider is better able to handle a patient with a cardiac emergency than a care provider without such training and/or experience. In some embodiments, one or more skill factors may be applied for a particular care provider to modulate the plurality of overload factors in a manner that reflects each individual care provider's training and/or experience.

In some embodiments, the network computing device may be configured to determine whether the plurality of overload factors modulated by the health condition of the care provider meets the overload condition. For example, a care provider who is sleep deprived, or has worked multiple shifts, or who is or was recently sick, who has worked many days in a row, and the like, may be more easily overloaded than a well-rested, healthy care provider.

In some embodiments, the network computing device may execute a trained model (e.g., a machine learning model) to determine whether the plurality of overload factors meets an overload condition for the care provider. In various embodiments, machine learning models are well suited to performing multi-factor or multi-variable analyses such as determining whether a plurality of overload factors meets and overload condition for a particular care provider. In some embodiments, the network computing device may apply the plurality of overload factors to an overload condition model that is configured to provide as an output whether the plurality of overload factors meets the overload condition.

In some embodiments, the overload condition model may be configured to provide as an output whether the plurality of overload factors meets the overload condition comprises applying one or more care provider skill factors to the overload condition model. In some embodiments, the overload condition model may be configured to provide as an output whether the plurality of overload factors modulated by the skill factors meets the overload condition. In some embodiments, the overload condition model may be configured to provide as an output whether the plurality of overload factors meets the overload condition comprises applying a health condition of the care provider to the overload condition model. In some embodiments, the overload condition model may be configured to provide as an output whether the plurality of overload factors modulated by the health condition of the care provider meets the overload condition.

As noted above, in response to determining that the plurality of overload factors meets the overload condition for the care provider, the network computing device may perform a mitigation operation. As noted above, the mitigation operation may provide the detection of the overload condition back as an input to a clinical workflow system. In some embodiments, the network computing device may send to a network computing device (e.g., a clinical workflow system) an instruction (or a request, or a recommendation) to reduce a frequency of communications (such as interrupt events) to the care provider. In some embodiments, the network computing device may request, instruct, or recommend that a frequency of communications be slowed to a particular rate, or below a particular rate. In some embodiments, the network computing device may request, instruct, or recommend that a frequency of communications be reduced to zero (e.g., “do not disturb”) for a period of time (e.g., 20 minutes) or until a specified time (e.g., until 3:30 AM).

In some embodiments, the network computing device may request, instruct, or recommend to adjust one or more tasks assigned to the care provider. For example, the network computing device may request, instruct, or recommend that a next task (or a number of tasks) be assigned to a different care provider. In some embodiments, the network computing device may request, instruct, or recommend that no further tasks be assigned to the care provider until the overload condition for that care provider has cleared.

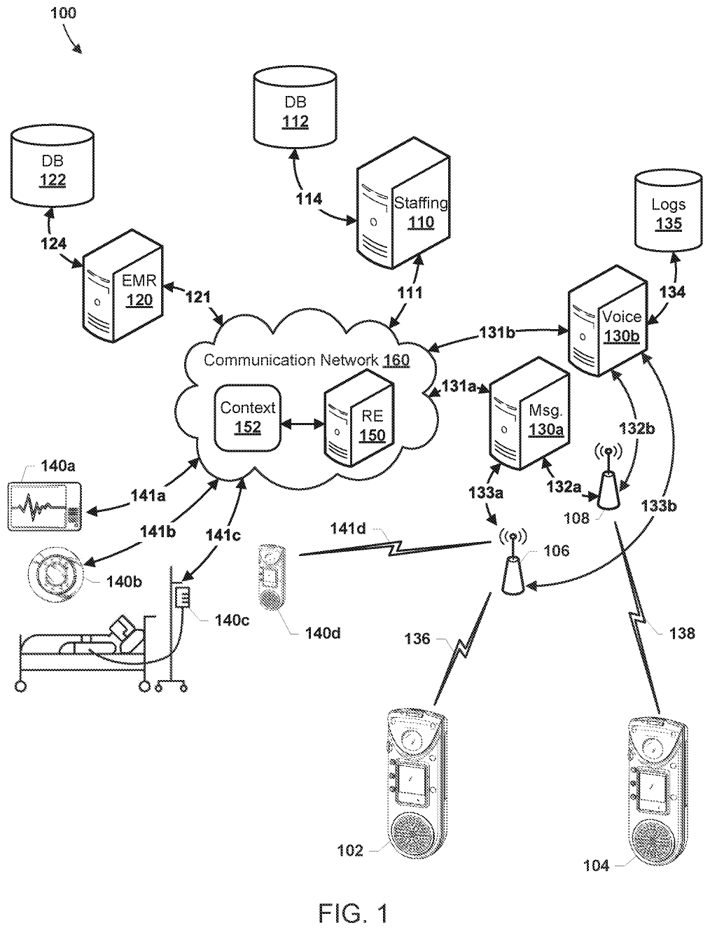

FIG. 1 illustrates a communication system 100 suitable for use with the various embodiments. The communication system 100 may include communication devices 102, 104, a staffing server 110, an electronic medical record (EMR) server 120, a messaging server 130a, a voice communications server 130b, one or more sensors/sources 140a-140d, and a rules engine 150. The various elements of the communication system 100 may be configured to communicate over a communication network 160 via wired or wireless communication links 111, 114, 121, 124, 131a, 131b, 132a, 132b, 133a, 133b, and 141a-141d. In some embodiments, one or more of the staffing server 110, the EMR server 120, the messaging server 130a, the voice communications server 130b, and the rules engine 150 may be configured as separate devices (e.g., server devices). In some embodiments, one or more of the staffing server 110, the EMR server 120, the messaging server 130a, the voice communications server 130b, and the rules engine 150 may be configured as separate logical services on one server or similar device.

The EMR server 120 may include one or more server computing devices configured to store, update, and transmit information such as patient-based data. The EMR server 120 may communicate over a wired or wireless communication link 124 with a database 122 configured to store data records. Patient-based data may include identifiers or codes indicating an identity of a patient, health care personnel associated with the patient (e.g., physician, specialist, hospitalist, nurse, etc.), patient location information (e.g., room, bed, wing, building), a status of the patient (e.g., discharged, admitted, etc.), and other suitable information.

The EMR server 120 may transmit messages (e.g., in HL7 or another suitable format) including patient-based data via one or more information feeds. In some embodiments, the EMR server 120 may transmit the messages on the occurrence of an event that changes the patient-based data at the EMR server 120. For example, the EMR server 120 may transmit a message that indicates a patient identifier and a room identifier in response to the patient corresponding to the patient identifier being admitted to the hospital and being assigned to a room corresponding to the room identifier.

In some embodiments, the EMR server 120 may be connected to or otherwise may utilize a system capable of sending and receiving HL7 version 2.3 messages (e.g., admit, discharge, transfer (ADT) messages), such as messages that include a role (or “ROL”) segment that indicates care team assignment information. The EMR server 120 may transmit information (e.g., in HL7, ADT messaging, or another suitable format) via one or more information feeds (e.g., to the rules engine 150).

The staffing server 110 may be one or more server computing devices configured to at least synchronize care team assignment data from different systems related to the hospital. The staffing server 110 may communicate over a wired or wireless communication link 114 with a database 112 configured to store data records. The staffing server 110 may transmit information (e.g., in HL7, ADT messaging, or another suitable format) via one or more information feeds (e.g., to the rules engine 150).

In some embodiments, the staffing server 110 may be configured to continually receive data (e.g., via the communication network 160) from the EMR server 120, the messaging server 130a, the voice communications server 130b, and/or other systems that indicate staffing changes (e.g., to care teams associated with the various patients, locations, and/or shifts of the hospital). For example, the staffing server 110 may receive subscription messages from the voice communications server 130b indicating when particular nurses of the hospital log-in or out of a shift and/or HL7 messages from the EMR server 120 that indicate when a particular patient's data changes (e.g., assigned to a new bed, room, specialist doctor, etc.). The data records may include records related to the various patients admitted to a hospital and/or the various care teams active in the hospital, and may be accessed to obtain a data record indicating the last known nurse, nurse assistant, bed, wing, building, physician, specialist, and hospitalist for a particular patient identifier.

The messaging server 130a may include one or more server computing devices configured to control various messages sent between the communication devices 102, 104 via access points 106, 108. In some embodiments, the messaging server 130a may be configured to control messages sent to the communication devices 102, 104 from the rules engine 150.

The voice communications server 130b may include one or more server computing devices configured to control various voice calls placed between the communication devices 102, 104 via wireless access points 106, 108. In some embodiments, the voice communications server 130b may include a signaling gateway service to facilitate communications between and among the communication devices 102, 104 and the voice communications server 130b, such as login functions, voice call functions, and other suitable functions. In some embodiments, the signaling gateway service may be configured as a separate device (not illustrated).

As noted above, in various embodiments, the messaging server 130a and the voice communications server 130b may be configured as separate devices, or as logical functions in one device. In some embodiments, the messaging server 130a and the voice communications server 130b may transmit information in a suitable format via one or more information feeds (e.g., to the rules engine 150).

In operation, the communication system 100 may include a large number of communication devices and access points, illustrated as communication devices 102, 104, 140d and access points 106, 108 for conciseness. The communication devices 102, 104, 140d may communicate with an access point 106, 108 over wireless communication links 136, 138, 141d. The access points 106, 108 may communicate with the voice communications server 130b and the messaging server 130a over communication links 132a, 132b, 133a, 133b. The voice communications server 130b may control various messages and voice calls placed between the communication devices 102, 104, 140d. The voice communications server 130b may communicate over a wired or wireless communication link 134 with a logs database 135 configured to store logs and other data records.

In some embodiments, the voice communications server 130b may be configured to provide information to the rules engine 150 via one or more information feeds. For example, the voice communications server 130b may store, update, and transmit at least shift-based and/or location-based data of the various care team assignments of the hospital. The voice communications server 130b may also store, update, and transmit patient-related information, information related to the facility or environment, and other information. For example, the voice communications server 130b may receive messages from any of the communication devices 102, 104, 140d that indicate users of the communication devices 102, 104, 140d have logged-out of or logged-into a shift of working in a care team at the hospital. As another example, the voice communications server 130b may receive a message from a communication device 102, 104, 140d regarding the condition of a patient, equipment, a location, an environmental condition, or other suitable information. The voice communications server 130b may transmit information in a suitable format via one or more information feeds (e.g., to the rules engine 150).

The one or more sensors/sources 140a-140d may include one or more sensor devices to sense information about a patient, an environment, or other suitable information. The one or more sensors/sources 140a-140d may further include one or more sources information about a patient, an environment, or other suitable information, such as a bed exit monitor, a nurse call button/system, a video surveillance system, or another suitable source. For example, patient monitors 140a, 140c may include devices configured to monitor one or more patient conditions or vital signs.

Room sensors 140b may be configured to sense and provide information about one or more environmental conditions, or aspects of a person or object to which the sensor is attached (e.g., temperature, humidity, motion, door or window security, ambient light conditions, location, acceleration, orientation, etc.) In some embodiments, the one or more sensors/sources 140a-140d may transmit information in a suitable format via one or more information feeds to the rules engine 150.

Communication devices 102, 104, 140d may also function as a source of clinical or call context information, such as identifying users of the devices (i.e., caregivers) in proximity to a patient. In some embodiments, the one or more sensors/sources 140a-140d may be configured with, or may communication with a device configured with, a processor and a wired or wireless communication capability to communicate sensed information in a suitable format over a wired or wireless communication links 141a-141d.

The rules engine 150 may include one or more server computing devices configured to receive clinical information via various information feeds from other network elements such as the staffing server 110, the EMR server 120, and the one or more sensors/sources 140a-140d. In various embodiments, the various network elements (e.g., the staffing server 110, the EMR server 120, and the one or more sensors/sources 140a-140d) may be configured to send information to the rules engine 150 in an unsolicited manner (e.g., without requiring a query or another message soliciting information).

By receiving information feeds from the other network elements, the rules engine 150 may avoid interfering with or otherwise altering the normal function and efficiency of the other network elements. For example, the rules engine 150 may not alter electronic medical records stored on the EMR server 120, but rather may receive information periodically provided by the EMR server 120 and stored on the rules engine 150.

The rules engine 150 may be configured to associate certain portions or element(s) of the clinical information with one or more event identifiers for use in generating call context information for an event identifier associated with a particular communication request (i.e., call). Further, the rules engine 150 may be configured to provide the call context information to a called communication device 102, 104, e.g., when a communication request is sent to a communication device 102, 104, as further described below.

The communication links 111, 114, 121, 124, 131a, 131b, 132a, 132b, 133b, 136, 138, and 141a-141d may include wired or wireless communication links. Wired communication links may include, for example, twisted pair cable, coaxial cable or fiber optic cable, or combinations thereof. Wireless communication links may include a radio frequency, microwave, infrared, or other similar signal. Wireless communication links may include a plurality of carrier signals, frequencies, or frequency bands, each of which may include a plurality of logical channels. For example, wireless communication links may be established over a Wi-Fi local area wireless communication network.

Other network elements may be present in a communication system 100 system to facilitate communications are omitted for clarity, including additional access points, processing nodes, routers, gateways, and other network elements, as well as physical and/or wireless data links for carrying signals among the various network elements.

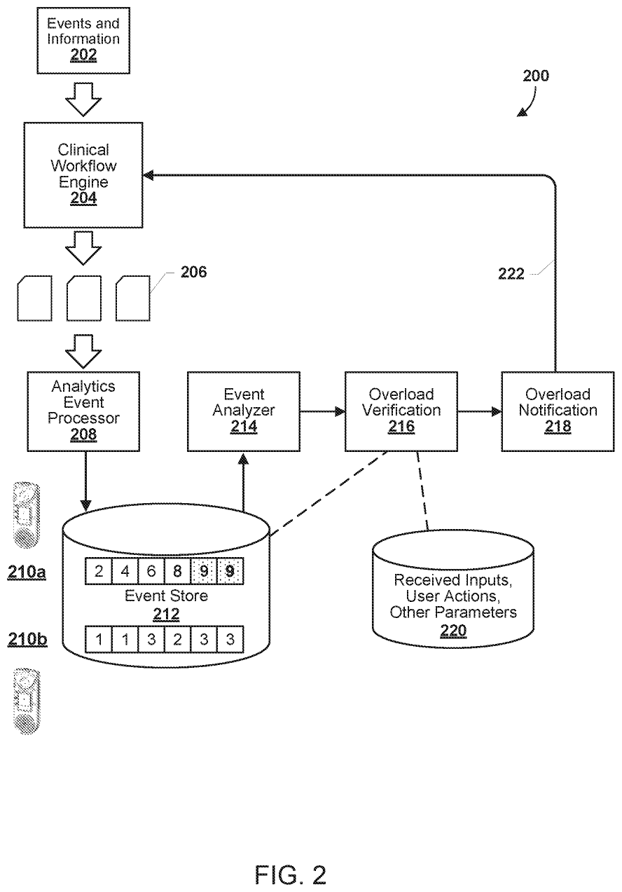

FIG. 2 is a conceptual block diagram illustrating operations of a workflow 200 suitable for use in some embodiments. With reference to FIGS. 1 and 2, the workflow 200 may be implemented in hardware components and/or software components of a network computing device (e.g., 110, 120, 130a, 130b, 150), the operation of any of which may be controlled by one or more processors (a “processor”). In some embodiments, the network computing device may include functions such as a clinical workflow engine 204, an analytics event processor 208, an event store 212, an event analyzer 214, an overload verification function 216, an overload notification function 218, and a data store 220 of received inputs, user actions, and other parameters.

In some embodiments, the clinical workflow engine 204 may receive events and information 202 from a variety of sources (e.g., 110, 120, 130a, 130b, 140a-140d). Based on at least the events and information 202, the clinical workflow engine 204 may generate one or more communications 206, which may include interrupt events. In some embodiments, the analytics event processor 208 may receive or monitor the communications 206.

In some embodiments, the analytics event processor 208 may identify one or more overload factors based on the communications 206. In some embodiments, the analytics event processor 208 may store in the event store 212 events that are associated with communication devices of care providers, such as communication device 210a associated with a first care provider and communication device 210b associated with a second care provider. In some embodiments, the events may be representations (e.g., numerical representations) of one or more overload factors.

In some embodiments, the analytics event processor 208 may employ a sliding window (e.g., a sliding window algorithm) to monitor communications to a care provider communication device over a period of time. Use of such a sliding window algorithm may reduce a use of computational resources and/or increase computational speed. In some embodiments, the analytics event processor 208 may initialize one or more counters for a sliding window length (e.g., time period). In some embodiments, one or more counters may be associated with a care provider. In some embodiments, in response to determining an overload factor for a care provider, the analytics event processor 208 may add a score for the determined overload factor to a counter associated with the care provider. In some embodiments, the analytics event processor 208 may associated a weight value with the score for the determined overload factor. In some embodiments, the analytics event processor 208 may add a weighted score for the determined overload factor to a counter associated with the care provider. In such embodiments, the analytics event processor 208 may rapidly generate a weighted total for each care provider (e.g., for the sliding window time period).

As an example implementation, the event store 212 may include a series of weighted counts of events for each communication device (e.g., for the communication device 210a and the communication device 210b, respectively). In this example, each illustrated number represents a weighted count or weighted score of events in a preceding sampling window period, in a sequence from older counts (on the right) to newer counts (on the left). For the communication device 210a, the weighted counts of events from oldest to newest are represented by 9, 9, 8, 6, 4, and 2. In this example, each number is a representation of overload factors associated with the communication device 210a. For the communication device 210b, the weighted counts of events from oldest to newest are represented by 3, 3, 2, 3, 1, and 1. In this example, each number is a representation of overload factors associated with the communication device 210b. Each series represents a sliding window that shifts to the left after a time-to-live timer (or another suitable timer) expires for each weighted score. In some embodiments, one or more of the weighted scores may be provided to the event analyzer 214 for analysis.

The event analyzer 214 may determine whether the plurality of overload factors meets an overload condition for a care provider. For example, based on representations of overload factors associated with the communication device 210a (which is associated with the first care provider), the event analyzer 214 may determine whether the overload factors associated with the communication device 210a meets an overload condition for the first care provider.

In some embodiments, the event analyzer 214 may send an indication of the overload condition to the overload verification function 216. In some embodiments, the overload verification function 216 may verify the determination that the overload factors meet the overload condition based on information stored in the event store 212. In some embodiments, the overload verification function 216 may verify the determination that the overload factors meet the overload condition based on information a received input or another user action or another parameter from data store 220. For example, the overload verification function 216 may send an indication of the overload condition for the first care provider to a communication device for a manager, supervisor, or another user, who may provide to the communication device an input verifying the overload condition.

In some embodiments, the overload verification function 216 (or the event analyzer 214) may send to the overload notification function an indication of the overload condition for the first care provider. In some embodiments, the overload notification function 218 may generate and send a notification 222 to the clinical workflow engine 204 indicating the overload condition for the first care provider.

In some embodiments, in response to receiving the notification 222, the clinical workflow engine 204 may perform a mitigation operation for the care provider. For example, the clinical workflow engine 204 may reduce a frequency of communications (such as interrupt events) to the care provider. As another example, the clinical workflow engine 204 may adjust one or more tasks assigned to the care provider. As another example, the clinical workflow engine 204 may redirect one or more communications and/or tasks to the second care provider.

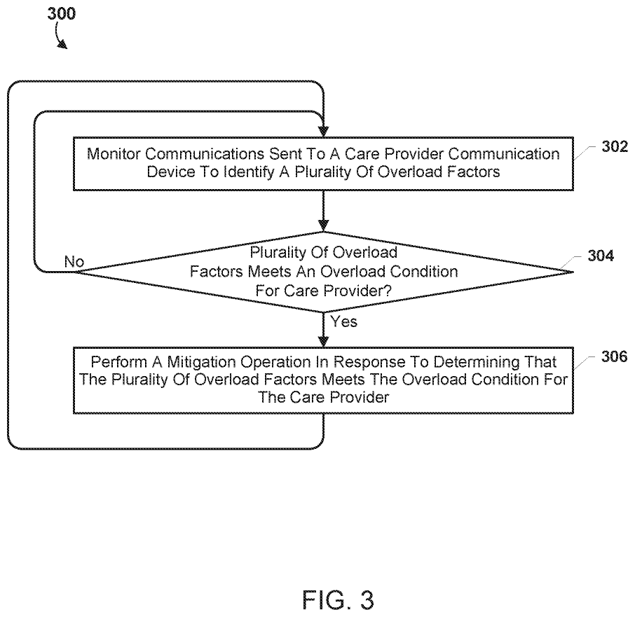

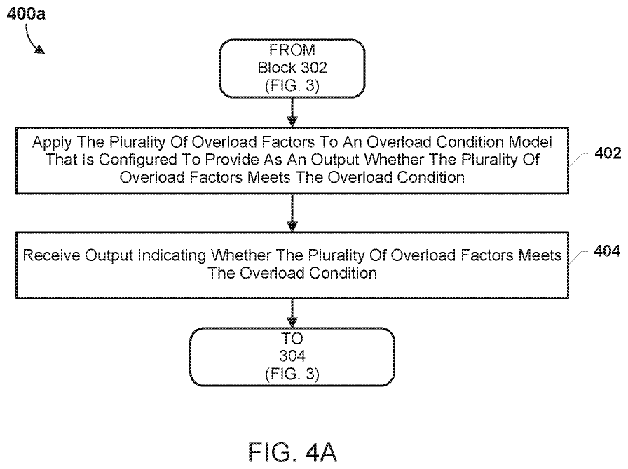

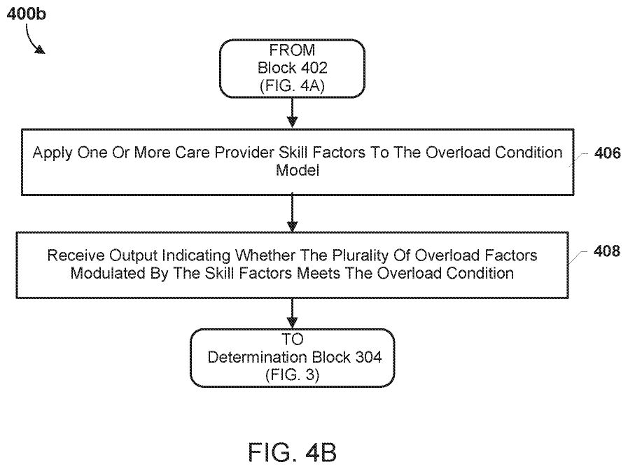

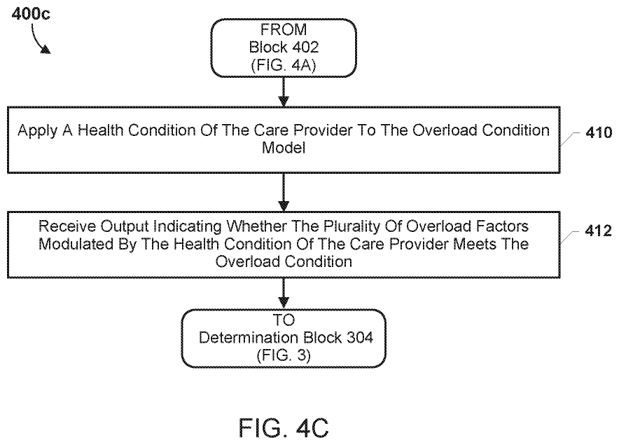

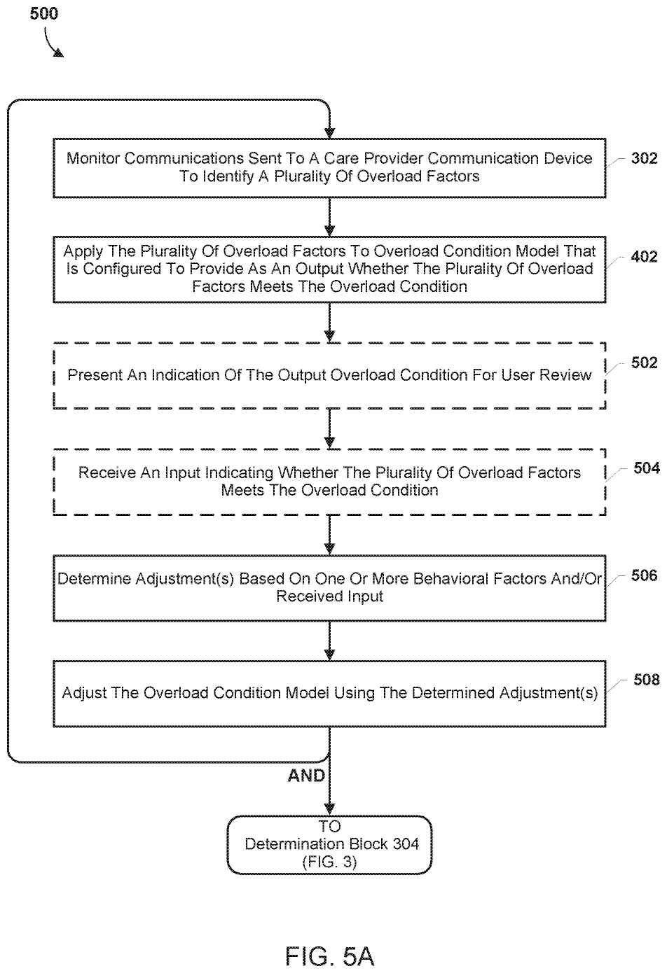

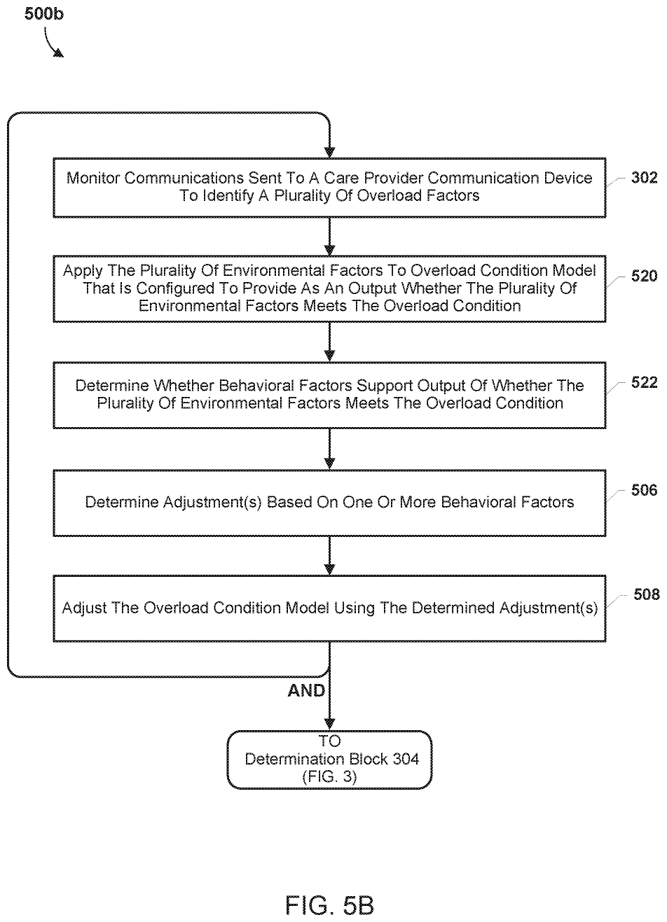

FIG. 3 is a process flow diagram illustrating a method 300 that may be performed by a processor of a network computing device for managing a care provider overload condition 300 according to various embodiments. With reference to FIGS. 1-3, the method 300 may be implemented in hardware components and/or software components of one or more server devices (e.g., 110, 120, 130a, 130b, 150) implementing one or more functions (e.g., 204, 208, 212, 214, 216, 218, 220), the operation of any of which may be controlled by one or more processors (a “processor”).

In block 302, the processor may monitor communications sent to a care provider communication device (e.g., 102, 104, 140d) to identify a plurality of overload factors. In some embodiments, the plurality of overload factors may include one or more interrupt events. In some embodiments, each interrupt event may be associated with a severity metric. In some embodiments, each interrupt event may be associated (e.g., by the processor) with a congruity metric that indicates a level of congruity of each interrupt event relative to one or more other interrupt events. In some embodiments, each interrupt event may be associated (e.g., by the processor) with an acuity metric that indicates a level of acuity of the interrupt event.

In some embodiments, the processor may identify one or more of a variety of parameters and conditions to identify the plurality of overload factors. In some embodiments, the processor may determine response times for receiving a response from a care provider communication device to communications. In some embodiments, the processor may monitor communications that are ignored or declined by the care provider communication device. In some embodiments, the processor may monitor communications that are responded to with a do not disturb instruction. In some embodiments, the processor may monitor a number of communications that have not been answered. In some embodiments, the processor may monitor how long the care provider has been working. In some embodiments, the processor may determine intervals between communications.

In determination block 304, the processor may determine whether the plurality of overload factors meets an overload condition for the care provider. In some embodiments, the processor may determine whether the plurality of overload factors meets an overload condition for the care provider using a rule-based determination or set of determinations. For example, the processor may determine that one or more of the plurality of overload factors (e.g., taken alone, or in some combination) meets the overload condition for the care provider. In some embodiments, the processor may apply the plurality of overload factors to an overload condition model that is configured to provide as an output whether the plurality of overload factors meets the overload condition.

In some embodiments, the processor may apply one or more provider skill factors to the overload factors. In such embodiments, the processor may determine whether the plurality of overload factors modulated by one or more care provider skill factors meets the overload condition. In some embodiments, the processor may apply a health condition of the care provider to the overload factors. In such embodiments, the processor may determine whether the plurality of overload factors modulated by the health condition of the care provider meets the overload condition.

In response to determining that the plurality of overload factors do not meet the overload condition for the care provider (i.e., determination block 304=“No”), the processor may continue to monitor communications sent to a care provider communication device in block 302.

In response to determining that the plurality of overload factors meets the overload condition for the care provider (i.e., determination block 304=“Yes”), the processor may perform a mitigation operation in response to determining that the plurality of overload factors meets the overload condition for the care provider in block 306. For example, the processor may reduce a frequency of communications (such as interrupt events) to the care provider. As another example, the processor may adjust one or more tasks assigned to the care provider. In some embodiments, the processor may send information (which may include an instruction) to a network computing device to reduce a frequency of communications to the care provider. In some embodiments, the processor may send information (which may include an instruction) to a network computing device to adjust one or more tasks assigned to the care provider.

The processor may repeat the operations of blocks 302-306 from time to time.

FIGS. 4A-4C are process flow diagrams illustrating operations 400a-400c that may be performed by a processor of a network computing device as part of the method 300 for managing a care provider overload condition according to various embodiments. With reference to FIGS. 1-4C, the operations 400a-400c may be implemented in hardware components and/or software components of one or more server devices (e.g., 110, 120, 130a, 130b, 150) implementing one or more functions (e.g., 204, 208, 212, 214, 216, 218, 220), the operation of any of which may be controlled by one or more processors (a “processor”).

Referring to FIG. 4A, following the operations of block 302 of the method 300 (FIG. 3), the processor may apply one or more care provider skill factors to the overload condition model in block 402. In some embodiments, the processor may apply the plurality of overload factors to an overload condition model that is configured to provide as an output whether the plurality of overload factors meets the overload condition.

In block 404, the processor may receive an output from the overload condition model indicating whether the plurality of overload factors meets the overload condition. In some embodiments, the output from the overload condition model may include an overload score that the processor may, for example, compare to an overload threshold to determine whether the plurality of overload factors meets an overload condition for the care provider. In some embodiments, the output from the overload condition model may include a decision, e.g., that the plurality of overload factors meets, or does not meet, the overload condition.

The processor may determine whether the plurality of overload factors meets an overload condition for the care provider in determination block 304 (FIG. 3), as described.

Referring to FIG. 4B, following the operations of block 402 (FIG. 4A), the processor may apply one or more care provider skill factors to the overload condition model in block 406.

In block 408, the processor may receive an output indicating whether the plurality of overload factors modulated by the skill factors meets the overload condition.

The processor may determine whether the plurality of overload factors meets an overload condition for the care provider in determination block 304 (FIG. 3), as described.

Referring to FIG. 4C, following the operations of block 402 (FIG. 4A), the processor may apply a health condition of the care provider to the overload condition model in block 410.

In block 412, the processor may receive an output indicating whether the plurality of overload factors modulated by the health condition of the care provider meets the overload condition.

The processor may determine whether the plurality of overload factors meets an overload condition for the care provider in determination block 304 (FIG. 3), as described.

FIGS. 5A and 5B are process flow diagrams illustrating operations 500a and 500b that may be performed by a processor of a network computing device as part of the method 300 for managing a care provider overload condition according to various embodiments. With reference to FIGS. 1-5B, the operations 500a and 500b may be implemented in hardware components and/or software components of one or more server devices (e.g., 110, 120, 130a, 130b, 150) implementing one or more functions (e.g., 204, 208, 212, 214, 216, 218, 220), the operation of any of which may be controlled by one or more processors (a “processor”). In some embodiments, the processor may perform the operations 500a and 500b to adjust the overload condition model on an individual basis, such as for a specific care provider.

Referring to FIG. 5A, in some embodiments, the processor may monitor communications sent to a care provider communication device (e.g., 102, 104, 140d) to identify a plurality of overload factors in block 302, as described.

In block 402, the processor may apply one or more care provider skill factors to the overload condition model, as described.

In some embodiments, the plurality of overload factors may include one or more environmental factors and one or more behavioral factors. In some embodiments, the environmental factors may include communications sent to the wireless device. The communications may include one or more interrupt events, each of which may be associated with a severity metric, a congruity metric, and/or an acuity metric, as described.

In some embodiments, the plurality of behavioral factors may include observable factors that are care-provider specific. In some embodiments, the behavioral factors may include response times for receiving a response from a care provider communication device to communications, communications that are ignored or declined by the care provider communication device, communications that are responded to with a do not disturb instruction, communications that have not been answered, how long the care provider has been working, and intervals between communications. The behavioral factors also may include receiving an indication from the wireless device that the care provider has provided an input to the wireless device that the care provider is going on a temporary break.

In optional block 502, the processor may present for user review an indication of the output from the overload condition model that the plurality of overload factors meets the overload condition. For example, the processor may display an indication or a prompt on a display of the wireless device soliciting input from the care provider as to whether the care provider is actually overloaded. Additionally or alternatively, in some implementations, the processor may display an indication or a prompt on a display of a wireless device (or another device) soliciting input from a supervisor of the care provider as to whether the care provider is actually overloaded. For example, while the processor may display an indication or a prompt on a display of the wireless device in an attempt to receive an input from the care provider, the care provider may be too overloaded to respond to the solicitation. In some implementations, the processor may display an indication or a prompt on a display of both the care provider's wireless device and the care provider's supervisor's wireless device. In some implementations, the processor may display an indication or a prompt on a display of the care provider's wireless device, and if the care provider does not respond within a threshold period of time, the processor may display an indication or a prompt on a display of the care provider's supervisor's wireless device.

In optional block 504, the processor may receive an input indicating whether the plurality of overload factors meets the overload condition. For example, the processor may receive such input using an input device (e.g., a touchscreen, button, etc.) of the wireless device. In some embodiments, the input may include a simple yes/no indication of whether the care provider feels overloaded. Additionally or alternatively, in some embodiments, the input may be more complex, such as an indication on a scale (e.g., from 1-10) of how overloaded the care provider feels.

In block 506, the processor may determine one or more adjustments to be made to the overload condition model based on one or more of the behavioral factors and/or the input received from the care provider. In various embodiments, by determining the adjustments using the behavioral factor(s) observed about the care provider and/or the input received from the care provider, the processor may adjust the overload condition model to be more reflective of the care provider's specific characteristics and capabilities. In some embodiments, the processor may determine whether the overload condition model has determined accurately whether the plurality of overload factors meets the overload condition. In some embodiments, the processor may determine an extent or degree to which the overload condition model is inaccurate or accurate (e.g., too sensitive, or not sensitive enough, and to what extent or degree). As one example, the overload condition model may output that the plurality of overload factors does not meet the overload condition, but the behavioral factors observed about the care provider indicate that the care provider is in fact overloaded. For example, the care provider's response times to communications may be over a threshold or increasing, a number of communications that are ignored or declined by the care provider communication device may be over a threshold or increasing, the communication device has indicated that the care provider is taking a break a threshold number (or an increasing number) of times, and so forth. As another example, the overload condition model may output that the plurality of overload factors meets the overload condition, but the input received by the wireless device indicates that the care provider does not feel overloaded. Based on one or more of the behavioral factors and/or the input received from the care provider, the processor may determine one or more adjustments to be made to the overload condition model.

In block 508, the processor may adjust the overload condition model using the one or more determined adjustments. In some embodiments, the processor may adjust one or more weight values of the overload condition. In some embodiments, a weight value may be applied to a severity metric, a congruity metric, and/or an acuity metric. In some embodiments, the overload condition model may adjust one or more care provider skill factors, which may be used to modulate the overload factors, as described.

In some embodiments, the processor may adjust the overload condition model to more accurately determine whether the overload factors are approaching the overload condition. In this manner, the overload condition model may be configured to provide an output anticipating whether the overload factors will meet the overload condition. In some embodiments, it may be more beneficial to determine that a care provider is trending toward being overloaded, or may soon become overloaded, rather than determining that a care provider is already overloaded. In some embodiments, in response to receiving an output anticipating that the overload factors for a particular care provider will meet the overload condition, the processor may generate and send a notification (e.g., 222) to a clinical workflow engine (e.g., 204) indicating the overload condition for the care provider. In response to the notification, the clinical workflow engine may adjust one or more tasks assigned to the care provider. In this manner, the processor may dynamically monitor one or more care providers, such as during a shift or another suitable work period, to anticipate when a care provider may become overloaded. Further, the processor may dynamically adjust communications to care providers and/or tasks assigned to care providers, to balance the communications sent to and task assignments of each care provider.

Following the performance of the operations of block 508, the processor may monitor communications sent to a care provider communication device to identify a plurality of overload factors in block 302, as described. Additionally, the processor may determine whether the plurality of overload factors meets an overload condition for the care provider in determination block 304, as described.

Referring to FIG. 5B, in some embodiments, the processor may automatically determine one or more adjustments to be made to the overload condition model based on one or more of the behavioral factors and/or the input received from the care provider.

In some embodiments, the processor may monitor communications sent to a care provider communication device (e.g., 102, 104, 140d) to identify a plurality of overload factors in block 302, as described.

In block 520, the processor may apply the plurality of environmental factors to the overload condition model that is configured to provide as an output whether the plurality of environmental factors meets the overload condition.

In block 522, the processor may determine whether the behavioral factors support the output of the overload condition model of whether the plurality of environmental factors meets the overload condition. For example, as noted above, the overload condition model may output that the plurality of overload factors does not meet the overload condition, but the behavioral factors observed about the care provider indicate that the care provider is in fact overloaded. As another example, the overload condition model may output that the plurality of overload factors meets the overload condition, but the behavioral factors do not indicate that the care provider is actually overloaded. For example, the care provider's response times to communications may be unchanged (or decreasing), a number of communications that are ignored or declined by the care provider communication device may be unchanged (or decreasing), and the like.

In block 506, the processor may determine one or more adjustments to be made to the overload condition model based on one or more of the behavioral factors and/or the input received from the care provider, as described.

In some embodiments, the processor may employ the operations 500a and 500b together or separately. In some embodiments, the processor may utilize the operations 500a earlier, such as to obtain baseline or initial information about a care provider, and the processor may later switch to using the operations 500b to make continual updates and adjustments to (i.e., to “fine tune”) the overload condition model for the particular care provider over time.

FIG. 6 illustrates a communication device 600 suitable for use in various embodiments. With reference to FIGS. 1-6, in some embodiments, the communication device 600 may include a voice communications badge device. The communication device 600 may include a housing 602 that encloses various components. The communication device 600 may include a microphone 610, a speaker 606, and a display device 604 such as a liquid crystal display (LCD). Various information may be displayed on the display device 604, such as data for reviewing text messages and pages received by the communication device 600 and/or data to facilitate the operation of the communication device 600. The microphone 610 and speaker 606 may also be used for voice communications. In some embodiments, the voice communication device 600 may further include an amplifier that amplifies the signals provided to/from the microphone and speaker.

The communication device 600 may further include an input device 614 that permits a user to configure and operate the communication device 600. In some embodiments, the input device 614 may be a jog switch that may be a spring-loaded compound-action switch that supports three momentary actions. In such embodiments, the switch may be pressed inwards as an ordinary push button. In some embodiments, the input device 614 may also be rotated in either direction and/or may be a touch button location in particular location (e.g., on the front of the communication device 600) that may be pushed or touched to activate the same functions and operations being activated by the jog switch. The communication device 600 may also include an on/off switch 616 and a status indicator (e.g., a light emitting diode (LED) that may be capable of displaying one or more different colors to signal the operational status of the communication device 600, etc.). In some embodiments, the communication device 600 may optionally include a headset jack that enables the user to plug in an external microphone/speaker headset, such as an ear bud.

Internally, the communication device 600 may include a central processing unit (CPU) or processor 650 that controls the operation of the components of the communication device 600. For example, the processor 650 may control the operations of the microphone 610 and the speaker 606 so that the communication device 600 may exchange voice communications, commands, and/or responses with remote devices (e.g., a voice communications server, etc.). The communication device 600 may further include a non-volatile memory device 652 so that data stored in the communication device 600 (such as settings, messages, and other data structures) are not lost when the communication device 600 is powered down. For example, the non-volatile memory device 652 may be a storage unit or other memory device configured to store at least a factory-assigned a unique physical media access control (MAC) address or unique wireless device address. The communication device 600 may also include a wireless transceiver 654 (e.g., an appropriate strength 802.5 transceiver, etc.) and an antenna 656 that may be used for wireless communications with various access points or with other devices (e.g., other communication devices, etc.). In some embodiments, the antennae 656 may be built into an exterior clip of the communication device 600 or may reside completely within the housing 602 of the communication device 600.

The communication device 600 may further include a pager receiver 660 that operates with the antenna 656 to receive text messages/pages within the coverage of any global paging service network. The communication device 600 may further comprise a digital signal processor (DSP) 662 and an audio codec 664 for processing incoming speech from the microphone 610 and for generating the voice signals generated by the speaker 606. For example, the DSP 662 and audio codec 664 may be capable of compressing digital voice data to reduce the amount of digital data used to communicate the voice commands to the server. The communication device 600 may include a power source 658, such as a removable, rechargeable battery that may include protection and charge management circuitry to prevent over-charging. For example, the energy source 658 may be a replaceable, rechargeable lithium polymer or lithium ion battery that fits on or in the housing 602. The various components may be connected via a bus or other similar linkage or connectivity.

Exemplary descriptions of various voice communications badge devices suitable for use in various embodiments may also be found in commonly-held patent applications, including U.S. Pat. No. 6,892,083 entitled “Voice-Controlled Wireless Communications System and Method,” U.S. Pat. No. 8,098,806 entitled “Non-User-Specific Wireless Communication System and Method,” and U.S. Design Patent D679,673, the content of all of which are incorporated herein for descriptions of various communication device components.

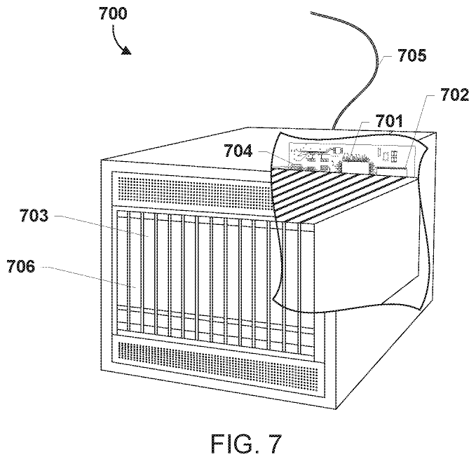

FIG. 7 illustrates a server device 700 suitable for use in various embodiments. With reference to FIGS. 1-7, various embodiments may employ the server device 700 as a network element of a communication system (e.g., the communication system 100). Examples of network elements that may be implemented in a server device, or as a logical service in a server device, include the staffing server 60, the EMR server 60, the messaging server 130a, the voice communications server 130b, and the rules engine 150. The server device 700 may include a processor 701 coupled to volatile memory 702 and a large capacity nonvolatile memory, such as a disk drive 703. The server device 700 may also include a peripheral memory access device such as a floppy disc drive, compact disc (CD) or digital video disc (DVD) drive 706 coupled to the processor 701. The server device 700 may also include network access ports 704 (or interfaces) coupled to the processor 701 for establishing data connections with a network 705, such as the Internet and/or a local area network coupled to other system computers and servers. The server device 700 may be coupled via a wired communication link 705 to one or more wireless access points (e.g., 106, 108). The server device 700 may include additional access ports, such as USB, Firewire, Thunderbolt, and the like for coupling to peripherals, external memory, or other devices.

The various processors described herein may be any programmable microprocessor, microcomputer or multiple processor chip or chips that can be configured by software instructions (applications) to perform a variety of functions, including the functions of the various embodiments described herein. In the various devices, multiple processors may be provided, such as one processor dedicated to wireless communication functions and one processor dedicated to running other applications. Typically, software applications may be stored in internal memory before they are accessed and loaded into the processors. The processors may include internal memory sufficient to store the application software instructions. In many devices the internal memory may be a volatile or nonvolatile memory, such as flash memory, or a mixture of both. For the purposes of this description, a general reference to memory refers to memory accessible by the processors including internal memory or removable memory plugged into the various devices and memory within the processors.

The foregoing method descriptions and the process flow diagrams are provided merely as illustrative examples and are not intended to require or imply that the operations of the various embodiments must be performed in the order presented. Accordingly, the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The hardware used to implement the various illustrative logics, logical operations, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more exemplary embodiments, the functions described may be implemented in hardware, software, firmware, or any combination thereof. If implemented in software, the functions may be stored on or transmitted over as one or more instructions or code on a non-transitory processor-readable, computer-readable, or server-readable medium or a non-transitory processor-readable storage medium. The operations of a method or algorithm disclosed herein may be embodied in a processor-executable software module or processor-executable software instructions which may reside on a non-transitory computer-readable storage medium, a non-transitory server-readable storage medium, and/or a non-transitory processor-readable storage medium. In various embodiments, such instructions may be stored processor-executable instructions or stored processor-executable software instructions. Tangible, non-transitory computer-readable storage media may be any available media that may be accessed by a computer. By way of example, and not limitation, such non-transitory computer-readable media may comprise RAM, ROM, EEPROM, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of non-transitory computer-readable media. Additionally, the operations of a method or algorithm may reside as one or any combination or set of codes and/or instructions on a tangible, non-transitory processor-readable storage medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

1. A computing device, comprising:
a memory; and
a processor coupled to the memory and configured with processor-executable instructions to:
monitor communications sent to a care provider communication device to identify a plurality of overload factors;
determine whether the plurality of overload factors meets an overload condition for the care provider; and
perform a mitigation operation in response to determining that the plurality of overload factors meets the overload condition for the care provider.

2. The computing device of claim 1, wherein the processor is further configured with processor-executable instructions such that the plurality of overload factors includes one or more interrupt events, wherein each interrupt event is associated with a severity metric.

3. The computing device of claim 2, wherein the processor is further configured with processor-executable to associate with each interrupt event at least one metric selected from the group consisting of (i) a congruity metric indicating a level of congruity of each interrupt event relative to one or more other interrupt events and (ii) an acuity metric indicating a level of acuity of the interrupt event.

4. (canceled)

5. The computing device of claim 1, wherein monitoring communications sent to a care provider communication device to identify a plurality of overload factors the processor comprises at least one of (i) determining response times for receiving a response from a care provider communication device to communications, (ii) monitoring communications that are ignored or declined by the care provider communication device, (iii) monitoring communications that are responded to with a do not disturb instruction, (iv) monitoring a number of communications that have not been answered, (v) monitoring how long the care provider has been working, or (vi) determining intervals between communications to or from the care provider communication device.

6-10. (canceled)

11. The computing device of claim 1, wherein determining whether the plurality of overload factors meets an overload condition for the care provider comprises determining whether the plurality of overload factors modulated by one or more care provider skill factors or by a health condition of the care provider meets the overload condition.

12. (canceled)

13. The computing device of claim 1, wherein the processor is further configured with processor-executable instructions to apply the plurality of overload factors to an overload condition model that is configured to provide as an output whether the plurality of overload factors meets the overload condition.

14. The computing device of claim 13, wherein the processor is further configured with processor-executable instructions to apply one or more care provider skill factors to the overload condition model,
wherein the overload condition model is configured to provide as an output whether the plurality of overload factors modulated by the skill factors meets the overload condition.

15. The computing device of claim 13, wherein the processor is further configured with processor-executable instructions to apply a health condition of the care provider to the overload condition model,
wherein the overload condition model is configured to provide as an output whether the plurality of overload factors modulated by the health condition of the care provider meets the overload condition.

16. The computing device of claim 1, wherein performing a mitigation operation in response to determining that the plurality of overload factors meets the overload condition for the care provider comprises at least one of (i) sending information to a network computing device to reduce a frequency of communications to the care provider or (ii) sending information to a network computing device to adjust one or more tasks assigned to the care provider.

17. (canceled)

18. A method performed by a processor of a network computing device, comprising:
monitoring communications sent to a care provider communication device to identify a plurality of overload factors;
determining whether the plurality of overload factors meets an overload condition for the care provider; and
performing a mitigation operation in response to determining that the plurality of overload factors meets the overload condition for the care provider.

19. The method of claim 18, wherein the plurality of overload factors includes one or more interrupt events, wherein each interrupt event is associated with a severity metric.

20. The method of claim 19, further comprising associating with each interrupt event at least one metric selected from the group consisting of (i) a congruity metric indicating a level of congruity of each interrupt event relative to one or more other interrupt events and (ii) an acuity metric indicating a level of acuity of the interrupt event.

21. (canceled)

22. The method of claim 18, wherein monitoring communications sent to a care provider communication device to identify a plurality of overload factors comprises at least one of (i) determining response times for receiving a response from a care provider communication device to communications, (ii) monitoring communications that are ignored or declined by the care provider communication device, (iii) monitoring communications that are responded to with a do not disturb instruction, (iv) monitoring a number of communications that have not been answered, (v) monitoring how long the care provider has been working. (vi) determining intervals between communications to or from the care provider communication device

23-29. (canceled)

30. The method of claim 18, wherein determining whether the plurality of overload factors meets an overload condition for the care provider comprises applying the plurality of overload factors to an overload condition model that is configured to provide as an output whether the plurality of overload factors meets the overload condition.

31. The method of claim 30, wherein applying the plurality of overload factors to an overload condition model that is configured to provide as an output whether the plurality of overload factors meets the overload condition comprises at least one of (i) applying one or more care provider skill factors to the overload condition model, wherein the overload condition model is configured to provide as an output whether the plurality of overload factors modulated by the skill factors meets the overload condition and (ii) applying a health condition of the care provider to the overload condition model, wherein the overload condition model is configured to provide as an output whether the plurality of overload factors modulated by the health condition of the care provider meets the overload condition.

32. (canceled)

33. The method of claim 18, wherein performing a mitigation operation comprises at least one of (i) sending information to a network computing device to reduce a frequency of communications to the care provider and (ii) sending information to a network computing device to adjust one or more tasks assigned to the care provider.

34-51. (canceled)

52. A non-transitory processor-readable medium having stored thereon processor-executable instructions configured to cause a processing device in a computing device to perform operations comprising:
monitoring communications sent to a care provider communication device to identify a plurality of overload factors;
determining whether the plurality of overload factors meets an overload condition for the care provider; and
performing a mitigation operation in response to determining that the plurality of overload factors meets the overload condition for the care provider.

53. The non-transitory processor-readable medium of claim 52, wherein the stored processor-executable instructions are further configured to cause the processing device in the computing device to perform operations such that the plurality of overload factors includes one or more interrupt events, wherein each interrupt event is associated with a severity metric.
wherein the stored processor-executable instructions are further configured to cause the processing device in the computing device to perform operations further comprising associating with each interrupt event at least one of (i) a congruity metric indicating a level of congruity of each interrupt event relative to one or more other interrupt events or (ii) an acuity metric indicating a level of acuity of the interrupt event.

54-63. (canceled)

64. The non-transitory processor-readable medium of claim 52, wherein the stored processor-executable instructions are further configured to cause the processing device in the computing device to perform operations such that determining whether the plurality of overload factors meets an overload condition for the care provider comprises applying the plurality of overload factors to an overload condition model that is configured to provide as an output whether the plurality of overload factors meets the overload condition.

65-66. (canceled)

67. The non-transitory processor-readable medium of claim 52, wherein the stored processor-executable instructions are further configured to cause the processing device in the computing device to perform operations such that performing a mitigation operation comprises at least one of (i) sending information to a network computing device to reduce a frequency of communications to the care provider or (ii) sending information to a network computing device to adjust one or more tasks assigned to the care provider.

68. (canceled)
